(19) United States
(12) Patent Application Publication
(10) Pub. No.: US 2018/0028782 A1
(43) Pub. Date: Feb. 1, 2018

(54) METHOD FOR CONTROLLING MAGNETIC CATHETER BY USING MAGNETIC-FIELD-GENERATED MAGNETIC ANNULUS

(71) Applicant: NATIONAL CHENG KUNG UNIVERSITY, Tainan City (TW)

(72) Inventors: CHING-HSING LUO, TAINAN CITY (TW); MENG-DAR SHIEH, TAINAN CITY (TW); CHIEN-CHEN CHANG, TAINAN CITY (TW); MING-HUNG TSAI, TAINAN CITY (TW); HSIN-EN FANG, TAINAN CITY (TW); YI ZHANG, TAINAN CITY (TW); YANG-BEN LIN, TAINAN CITY (TW); WEN-HORNG YANG, TAINAN CITY (TW); MING-CHANG SHIH, TAINAN CITY (TW); CHENG-CHI TAI, TAINAN CITY (TW)

(21) Appl. No.: 15/219,605

(22) Filed: Jul. 26, 2016

(51) Int. Cl. A61M 25/01; A61B 1/00; A61B 17/00; A61B 34/00

(52) U.S. Cl. CPC A61M 25/0127; A61B 34/73; A61B 1/00133; A61B 1/00158; A61B 17/00234; A61B 2034/732; A61B 2017/00314; A61B 2017/00318

(57) ABSTRACT

A method for controlling a magnetic catheter by using a magnetic-field-generated magnetic annulus is disclosed. The magnetic catheter has a free end provided with a magnetic member. A resultant magnetic field between at least two magnets generates a magnetic annulus. The magnetic catheter is placed into the magnetic annulus, so that the magnetic member is affected by the magnetic force from the magnetic annulus to guide the magnetic catheter to perform a preset motion. The magnetic catheter has a flexible front section, so that the flexible section can perform a bending motion when led by the magnetic member. The resultant magnetic field is generated by arranging the two magnets with their like poles facing each other, so that the magnetic member is thrust when entering the magnetic annulus. This facilitates the bending motion of the flexible section.

### BACKGROUND OF THE INVENTION

### 1. Technical Field

The present invention relates to a method for controlling a magnetic catheter, and more particularly to a method of moving a magnetic catheter by applying an acting force thereon via an annular region, hereafter called magnetic annulus, which has a relative high magnetic flux density in a magnetic field and offsets from a center of the magnetic field.

### 2. Description of Related Art

Thanks to the development of modern medical technology, many physical issues used to rely only on conventional open surgery can now be well treated and controlled using interventional catheter or endoscopic technology. This change helps to free patients from the discomfort caused by surgical operation, and minimize the risk of surgical infection.

To well control a flexible catheter, magnetic control has become an increasingly interesting scientific field. For example, U.S. Pat. No. 6,311,082 titled “DIGITAL MAGNETIC SYSTEM FOR MAGNETIC SURGERY” discloses a device that controls a magnetic field, generated by plural electromagnets, in terms of strength and direction by varying the currents and directions of theses electromagnets, so as to maneuver magnetic catheters inside patients' bodies to bend, travel and rotate as needed. However, using plural electromagnets renders the prior-art device bulky and expensive.

The inventor of the present invention has extensive study in the field of remote magnetic control (RMC) technology and has previously filed Taiwan Patent Application No. 102140727 for a motion-controlling device for catheters and a magnetic motion-controlling system for endoscopic catheters, and Taiwan Patent Application No. 101443778 for a magnetically controlled endoscope system and its magnetic-controlling device.

However, by implementing these inventors in practical applications, the inventor has noted that the following issues to be addressed.

First, the lines of magnetic force in the magnetic field between two opposite magnets are of a concentric annulus pattern, and the magnetic flux density is not even through the magnetic field. As a result, once the catheter to be controlled enters an area where the magnetic flux density is low, the controlling effect is degraded.

Second, when the bending of the catheter is purely depending on the attraction generated in the magnetic field, high magnetic flux density is required. For providing such high magnetic flux density, the electromagnets need to be supplied with extremely high current, and this is often costly.

Third, the attraction generated in the magnetic field can only make the catheter between the two opposite magnets bend toward one of the magnets at a relatively limited bending angle.

### SUMMARY OF THE INVENTION

Hence, the objective of the present invention is to provide a method for controlling a magnetic catheter by using a magnetic-field-generated magnetic annulus. The magnetic catheter has its free end provided with a magnetic member, and the method comprises:

generating a resultant magnetic field between at least two magnets, in which an annular region that has a relative high magnetic flux density is defined as a magnetic annulus; entering the magnetic catheter into the magnetic annulus and therefore producing a magnetic force between the magnetic member and the magnetic annulus; and making the magnetic member lead the magnetic catheter to perform a preset motion under the magnetic force. It is noted that the boundary and the range of the magnetic annulus are dynamic and changed according to interaction between the magnetic member and the resultant magnetic field.

Preferably, the magnetic annulus of the resultant magnetic field is generated by arranging the at least two magnets with their like poles facing each other.

Preferably, the magnetic catheter has a front section that is a flexible section, and the preset motion is to make the flexible section perform a bending motion. In an embodiment the flexible section and a rear section of the magnetic catheter have different rigidities due to the fact that they are made of different materials. In another embodiment, the flexible section is a multi joint section having the free end provided with the magnetic member; moreover, the joints of the multi joint section each have a single bending degree of freedom, and the preset motion is to make the flexible section perform a bending motion in the direction of the bending degree of freedom.

Preferably, the flexible section is resilient, and the method further comprises when the flexible section performs the bending motion, gradually increasing or decreasing a strength of the resultant magnetic field in a certain ratio according to the resilient returning force generated, so as to adjust the magnetic force applied on the magnetic member and thereby control a bending angle of the flexible section.

Preferably, the method comprises the step of when the flexible section has performed the bending motion, gradually decreasing the strength of the resultant magnetic field, thereby reducing the magnetic force acting on the magnetic member and enabling the flexible section to gradually return by its resilience, and further comprises when the flexible section returns to a set angle, reversing the resultant magnetic field, thereby enabling the flexible section to return to an initial state thereof under the magnetic force.

Preferably, the method comprises the step of according to a selected target site, when the flexible section performs the bending motion, changing a position of the resultant magnetic field, or/and changing a direction of the resultant magnetic field, or/and changing a strength of the resultant magnetic field, and retaining the magnetic member within the magnetic annulus. Moreover, the direction and the strength of the resultant magnetic field is adjustable by changing relative positions of the at least two magnets.

Preferably, the at least two magnets are electromagnets, and the direction and the strength of the resultant magnetic field is adjustable by changing current intensities or/and current directions of the electromagnets.

Preferably, the method comprises controlling the magnetic catheter to perform a feeding motion or/and a rotating motion synchronously.

Preferably, the magnetic member is an axial magnet.

Preferably, the at least two magnets are permanent magnets or electromagnets. In the case that the at least two magnets are electromagnets, the magnetic annulus refers to a region within which the magnetic member is able to lead the magnetic catheter to perform an expected motion that matches for output currents of the electromagnets, such as bending to an expected level that matches for the output currents of the electromagnets.

Preferably, the magnetic catheter is a flexible endoscope.

With at least one of the features as described above, the following effects can be achieved:

1. By applying an acting force on the magnetic member via an annular region (magnetic annulus) that has a relative high magnetic flux density in a magnetic field, it is easier for the magnetic member to drive the flexible section to perform a bending motion.

2. When the magnetic member enters a magnetic annulus of a resultant magnetic field generated by repulsion existing between like poles of two magnets, thrust is exerted on a head portion of the magnetic member to facilitate the bending motion and increase the bending angle of the flexible section. Thus, when the resultant magnetic field is generated by electromagnets, the required power output of the electromagnets can be reduced.

3. The magnetic annulus of the resultant magnetic field is generated by repulsion existing between like poles of the two magnets so that the flexible section is allowed to bend in a direction that is perpendicular to the extending direction of the two magnets and therefore able to bend in different directions

4. In the process that the strength of the resultant magnetic field is gradually decreased to return the flexible section, by reversing the resultant magnetic field, the head portion of the magnetic member will be pulled under the acting force and thus lead the multi joint section return to its initial state.

### BRIEF DESCRIPTION OF THE DRAWINGS

FIG. 1A is an applied view of a first type of magnetic control according to the present invention.

FIG. 1B is another applied view of the first type of magnetic control according to the present invention.

FIG. 2A is an applied view of a second type of magnetic control according to the present invention.

FIG. 2B is another applied view of the second type of magnetic control according to the present invention.

FIG. 2C is still another applied view of the second type of magnetic control according to the present invention.

FIG. 3A is an applied view of a third type of magnetic control according to the present invention.

FIG. 3B is another applied view of the third type of magnetic control according to the present invention.

FIG. 3C is still another applied view of the third type of magnetic control according to the present invention.

FIG. 4A is an applied view of a fourth type of magnetic control according to the present invention.

FIG. 4B is another applied view of the fourth type of magnetic control according to the present invention.

FIG. 4C is still another applied view of the fourth type of magnetic control according to the present invention.

FIG. 5A is an applied view of a fifth type of magnetic control according to the present invention.

FIG. 5B is another applied view of the fifth type of magnetic control according to the present invention.

FIG. 6 is a perspective view according to one embodiment of the present invention, showing a multi joint section of a magnetic catheter being disposed in a resultant magnetic field.

FIG. 7 is a side view showing the multi joint section of the magnetic catheter in FIG. 6.

FIG. 8 is a schematic drawing according to one embodiment of the present invention, showing that the multi joint section of the magnetic catheter performs a bending motion under the acting force produced from an annular region, in the resultant magnetic field, which has a relative high magnetic flux density, wherein the resultant magnetic field is generated between the like poles of the two magnets.

FIG. 9 is a schematic drawing according to one embodiment of the present invention, showing how the multi joint section bends in the case that the resultant magnetic field is not moved while the multi joint section of the magnetic catheter performs the bending motion.

FIG. 10 is a schematic drawing according to the embodiment in FIG. 9, representing the relationship between the current required and the bending angle in the case that the resultant magnetic field is not moved synchronously while the multi joint section of the magnetic catheter performs the bending motion.

FIG. 11 is a schematic drawing according to one embodiment of the present invention, showing how the multi joint section bends in the case that the magnetic member is retained within the magnetic annulus by moving the resultant magnetic field while the multi joint section of the magnetic catheter performs the bending motion.

FIG. 12 is a schematic drawing according to the embodiment in FIG. 11, representing the relationship between the current required and the bending angle in the case that the magnetic member is retained within the magnetic annulus by moving the resultant magnetic field while the multi joint section of the magnetic catheter performs the bending motion.

FIG. 13 is a schematic drawing according to one embodiment of the present invention, representing the relationship between the currents for the electromagnets and the bending angle of the magnetic catheter, in which the strength of the resultant magnetic field is gradually reduced until the direction of the resultant magnetic field is changed so as to return the multi joint section of the magnetic catheter.

FIG. 14 is a schematic view according to the embodiment in FIG. 13, showing how the multi joint section of the magnetic catheter is returned to its initial state while being pulled by the resultant magnetic field whose direction has been changed.

FIG. 15 is a perspective view of the magnetic catheter according to one embodiment of the present invention, depicting an exemplificative structure of the magnetic catheter for practical use.

FIG. 16 is another perspective view of the magnetic catheter of FIG. 15, showing how the magnetic catheter performs a bending motion.

FIG. 17 is a schematic view according to one embodiment of the present invention, showing how the elastic elements combined with the multi joint section of the magnetic catheter detect the actual bending angle.

FIG. 18 is a flow chart according to one embodiment of the present invention, explaining how the elastic elements combined with the multi joint section of the magnetic catheter detect the actual bending angle that is to be compared with the preset bending angle.

FIG. 19 is a schematic drawing illustrating the multi joint section of the magnetic catheter bending from the first side toward the second side.

FIG. 20 is a schematic drawing illustrating the multi joint section of the magnetic catheter bending from the second side toward the first side.

### DETAILED DESCRIPTION OF THE INVENTION

For further illustrating the means and functions on which the present invention achieves the certain objectives, the following description, in conjunction with the accompanying drawings and preferred embodiments, is set forth as below to illustrate the implement, structure, features and effects of the subject matter of the present invention.

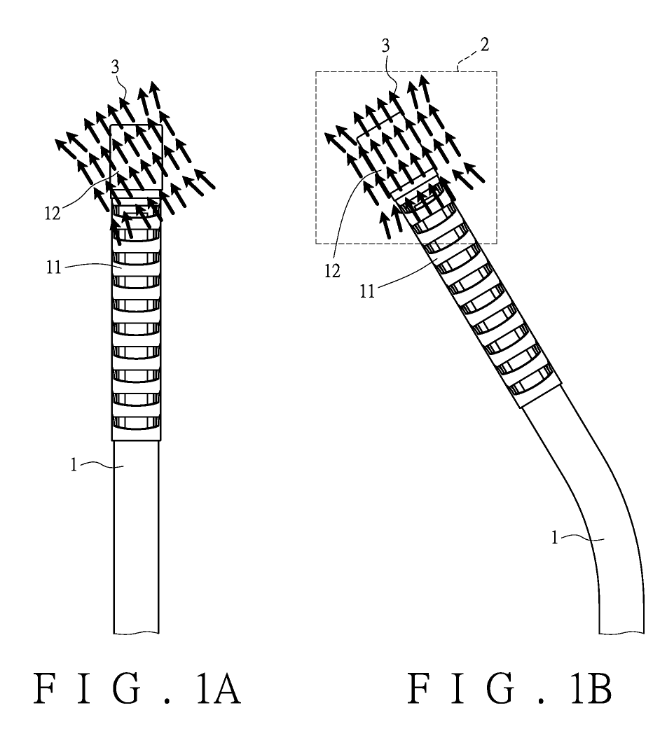

Referring to FIG. 1A, in the present embodiment, a magnetic catheter (1) has a front section formed as a flexible section. According to the present embodiment, the flexible section is a multi joint section (11) with each joint thereof having a single bending degree of freedom. At a free end of the multi joint section (11), a magnetic member (12) is provided. The magnetic member (12) is an axial magnet. Therein, the magnetic catheter (1) is capable of performing a feeding motion and a rotating motion along a linear first route. The first route is an extending route of the magnetic catheter (1).

In embodiments of the present invention, five types of magnetic control are applicable.

The first type is as shown in FIG. 1A and FIG. 1B.

In Step A, a target site (2) is set, as shown in FIG. 1B.

In Step B, at least two magnets are set opposite and separated from each other by a proper distance so as to form a resultant magnetic field (3). The resultant magnetic field (3) is applied to the multi joint section (11) of the magnetic catheter (1) and has a direction pointing toward the target site (2), while at this time the target site (2) is in a direction different from the direction of the bending degree of freedom.

In Step C, the magnetic catheter (1) is controlled not to perform the feeding motion and the rotating motion, and the magnetic member (12) is thus driven by the resultant magnetic field (3) to make the magnetic catheter (1) perform a declination, thereby making the free end of the multi joint section (11) point toward the target site (2).

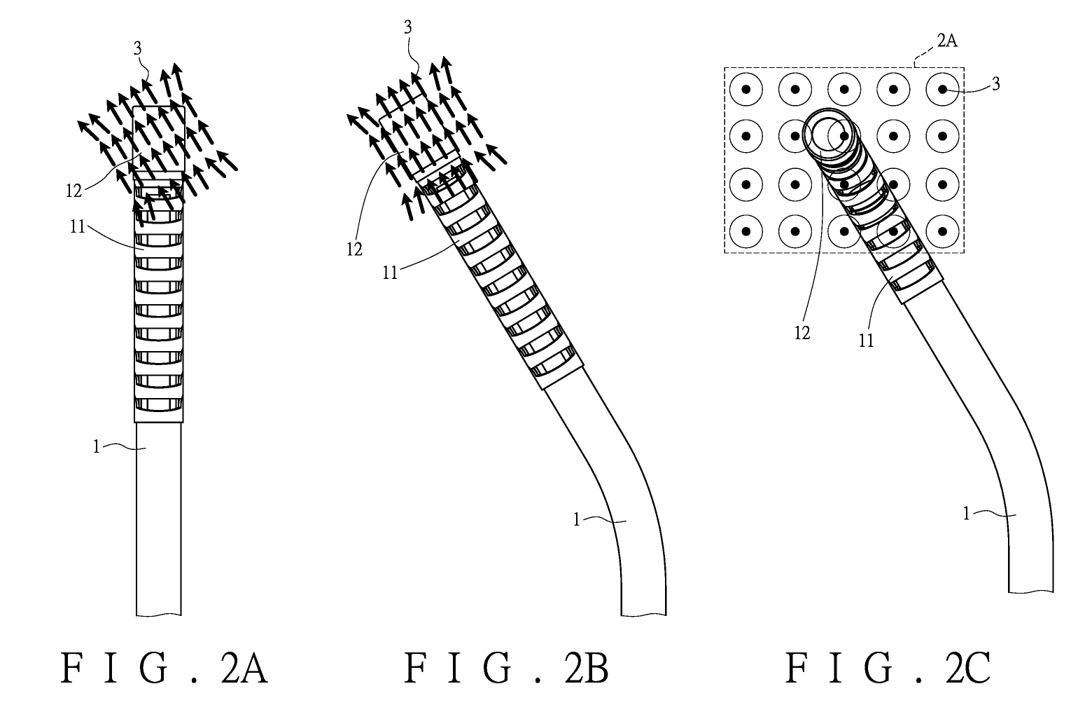

The second type is as illustrated in FIG. 2A through FIG. 2C.

This type has an addition step after the declination of the magnetic catheter (1) as described in the first type. The addition step, step D, involves changing the direction of the resultant magnetic field (3) again to make the resultant magnetic field (3) point toward the bending degree of freedom, so that the magnetic member (12) can be driven by the resultant magnetic field (3) to lead the multi joint section (11) to perform a bending motion along the bending degree of freedom. The multi joint section (11) can thereby be in a three-dimensional torsion state, as shown in FIG. 2C, with the free end thereof pointing toward another target site (2A).

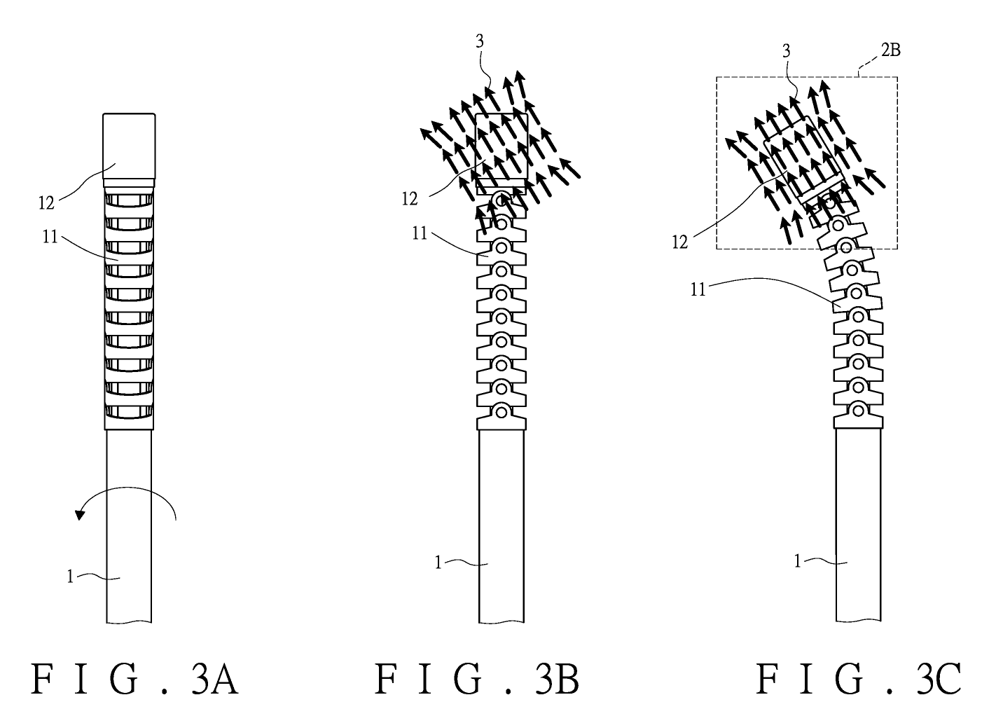

The third type is as illustrated in FIG. 3A through FIG. 3C.

In Step A, the magnetic catheter (1) is such rotated that a target site (2B) is set in the direction of the bending degree of freedom of the multi joint section (11).

In Step B, the resultant magnetic field (3) is applied to the multi joint section (11) and has its direction pointing toward the target site (2B).

In Step C, the magnetic catheter (1) is controlled not to perform the feeding motion and the rotating motion, and the magnetic member (12) is thus driven by the resultant magnetic field (3) to make the multi joint section (11) of the endoscopic catheter (1) perform a bending motion along the bending degree of freedom, thereby making the free end point toward the target site (2B).

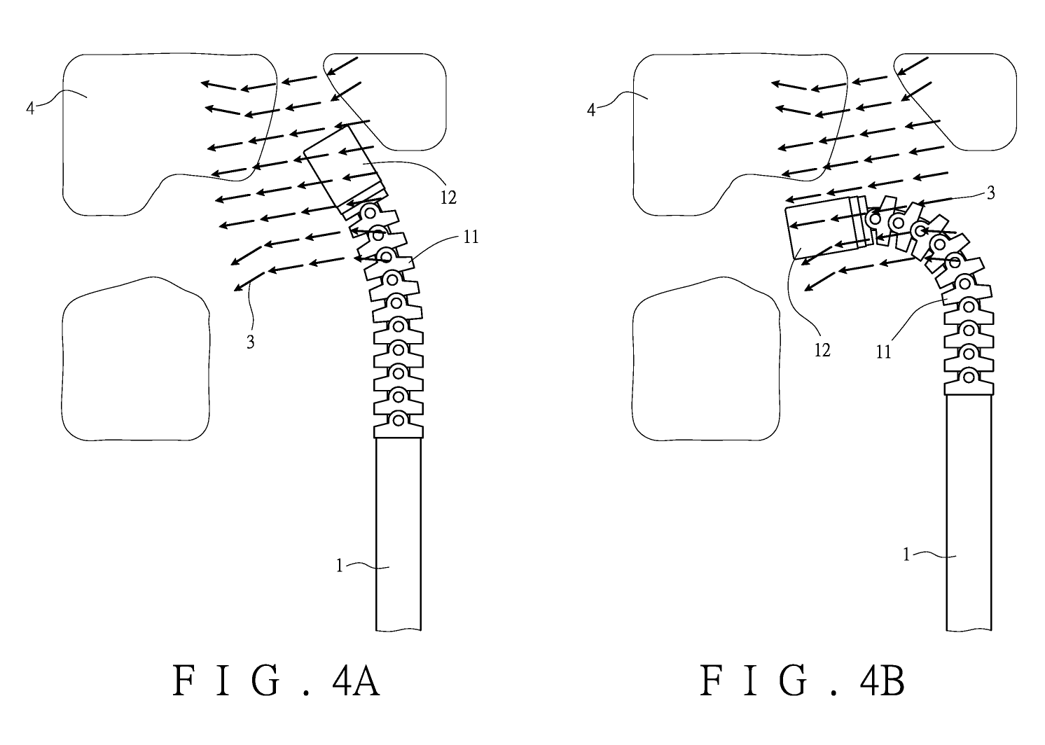

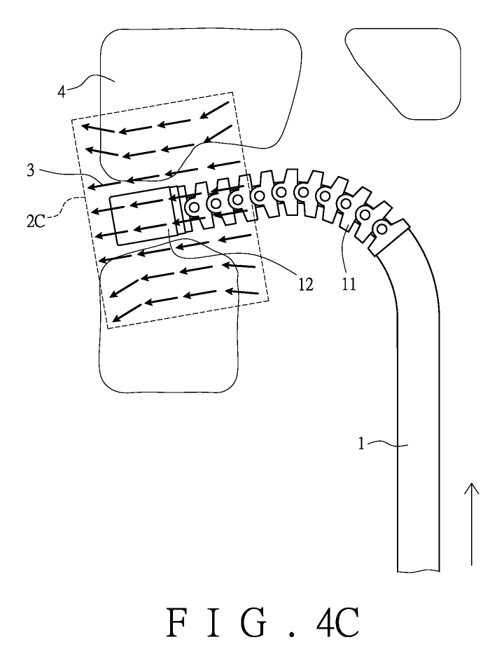

The fourth type is as illustrated in FIG. 4A through FIG. 4C.

In Step A, the magnetic catheter (1) enters a body cavity (4), and a target site (2C) is set. The target site (2C) is located in the direction of the bending degree of freedom of the multi joint section (11).

In Step B, the resultant magnetic field (3) is applied to the multi joint section (11) of the magnetic catheter (1) and has a direction pointing toward the target site (2C).

In Step C, the magnetic catheter (1) is controlled not to perform the feeding motion and the rotating motion, and the multi joint section (11) thus performs a bending motion to avoid obstacles.

In Step D, the resultant magnetic field (3) is moved while the magnetic catheter (1) is controlled to perform the feeding motion, so that the magnetic member (12) is driven by the resultant magnetic field (3) to control the free end of the multi joint section (11) to reach the target site (2C).

In addition to the method described above, by shifting the resultant magnetic field (3) and controlling the magnetic catheter (1) to perform the feeding motion, the free end of the multi joint section (11) can linearly advance toward and reach a desired target site.

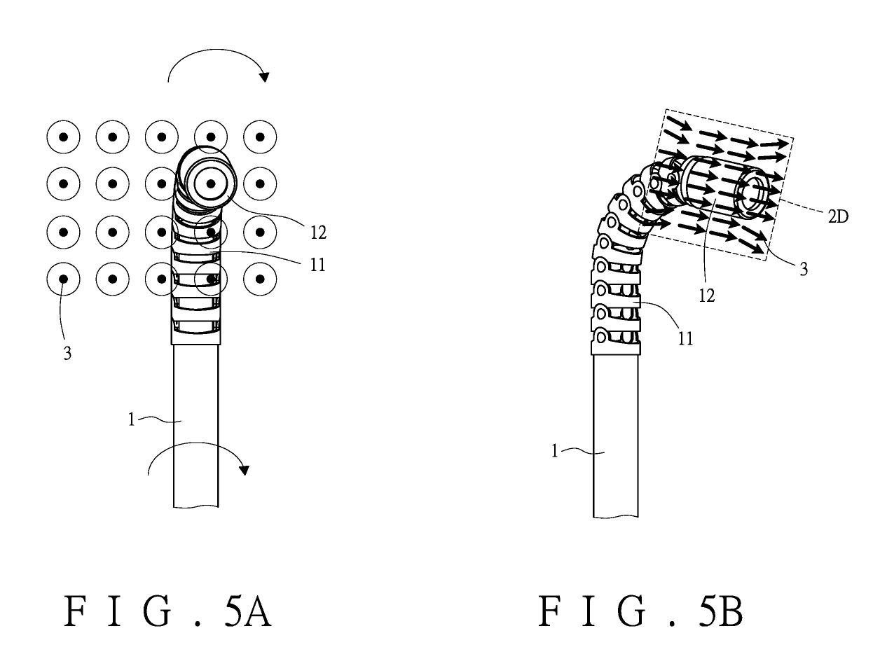

The fifth type is as illustrated in FIG. 5A and FIG. 5B.

In Step A, a target site (2D) is set.

In Step B, the resultant magnetic field (3) is applied to the multi joint section (11) of the magnetic catheter (1) and has a direction pointing toward the direction of the bending degree of freedom.

In Step C, the magnetic catheter (1) is controlled not to perform the feeding motion and the rotating motion, and the multi joint section (11) of the magnetic catheter (1) thus performs a bending motion in the direction of the bending degree of freedom.

In Step D, the resultant magnetic field (3) is rotated, and the magnetic catheter (1) is also rotated according to the direction of the resultant magnetic field (3), so that the direction of the resultant magnetic field (3) is aligned with the direction of the bending degree of freedom of the multi joint section (11), thereby driving the free end of the multi joint section (11) to point toward the target site (2D).

The application of the resultant magnetic field (3) and the synchronous control of the feeding and rotating motions of the magnetic catheter (1) jointly ensure that the free end of the multi joint section (11) can selectively point toward any one of the target sites (2)(2A)(2B)(2C)(2D). With the cooperation of the feeding and rotating motions of the magnetic catheter (1), the magnetic member (12) is prevented from becoming uncontrollable to the resultant magnetic field (3), which may otherwise causes unexpected operational errors when the resultant magnetic field (3) shifts or changes direction. More specifically, without synchronously feeding or rotating the magnetic catheter (1) according to the movement or the direction of the resultant magnetic field (3), the magnetic catheter (1) could be twisted and thus generate considerable resistance that hinders the multi joint section (11) from following the resultant magnetic field (3). The free end of the multi joint section (11) then could fail to reach the target site and even come out of the control of the resultant magnetic field (3). While the present invention is effective in overcoming this problem, the solution is not limited to that described above and can be designed by varying the resultant magnetic field (3) and feeding/rotating the magnetic catheter (1) according to any desired target site. The present invention thus provides interventional or endoscopic surgery with a method of reaching nidi quickly and precisely through magnetic control.

It is to be noted that instead of making the flexible section as the multi joint section (11), the present invention may have the flexible section and the rear section of the magnetic catheter (1) made of materials of different rigidities.

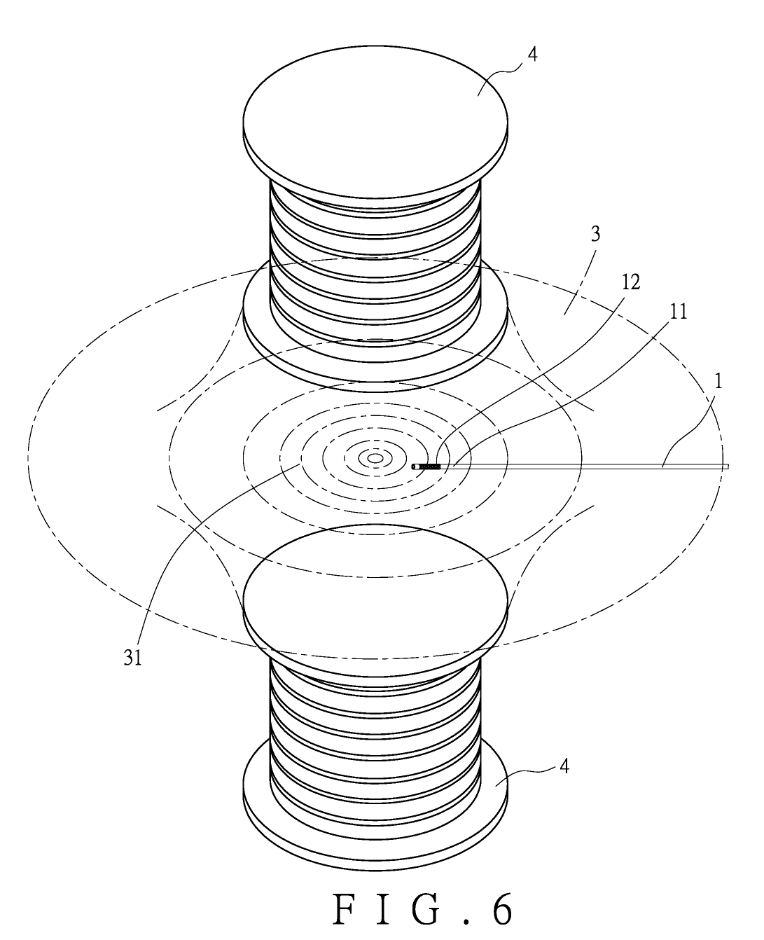

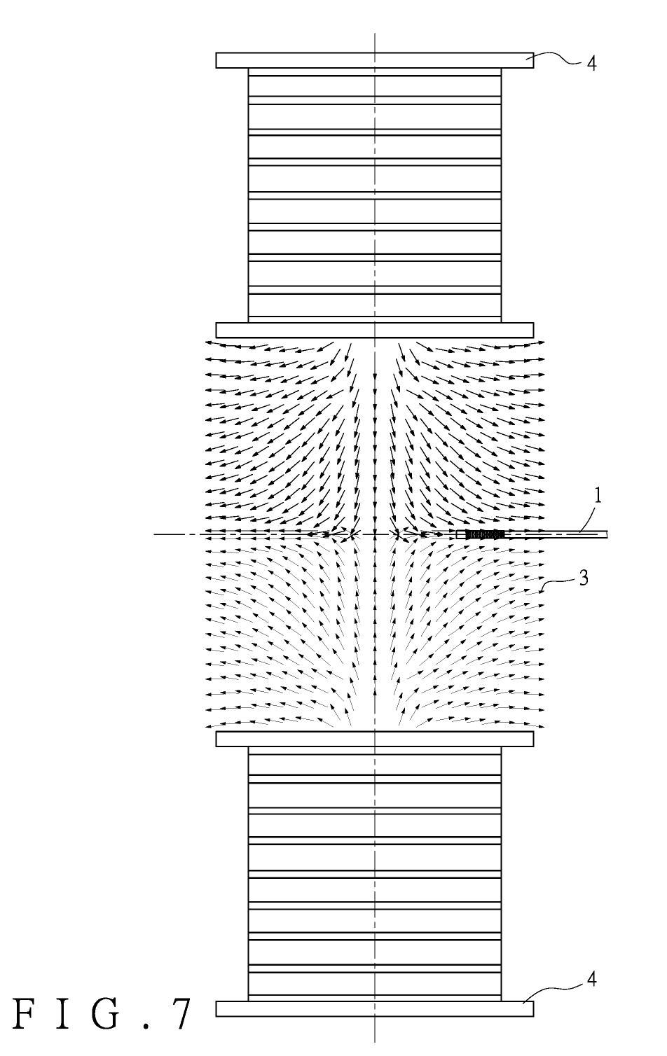

Referring to FIG. 6 through FIG. 8, the resultant magnetic field (3) is generated between two magnets, and an annular region therein is defined as a magnetic annulus (31). Generally, the magnetic annulus (31) refers to the region that has highest magnetic flux density on an acting plane, for example the region involving top 50 percent of the highest magnetic flux densities. When the multi joint section (11) of the magnetic catheter (1) enters the magnetic annulus (31) of the resultant magnetic field (3), a magnetic force is produced by the interaction between the magnetic member (12) and the magnetic annulus (31). Therein, the magnetic annulus (31) of the resultant magnetic field (3) is generated by the two magnets whose like poles face each other. For convenient control over the strength and direction of the resultant magnetic field (3), the magnets may be electromagnets (5).

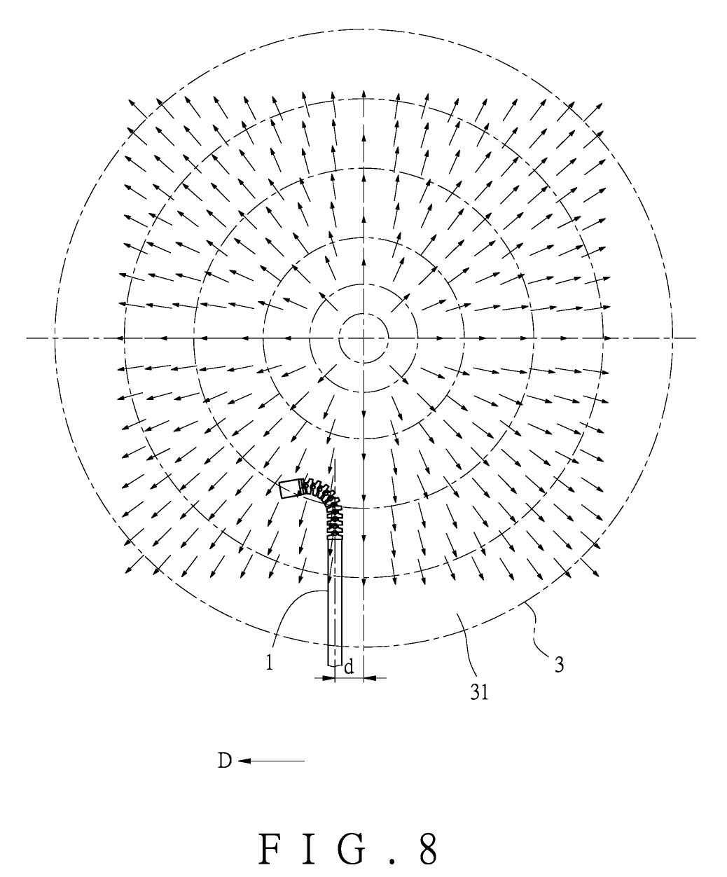

Referring to FIG. 8, the direction of the bending degree of freedom of the multi joint section (11) is pointed toward a desired direction (D). At this time, a magnetic force is produced by the interaction between the magnetic member and the magnetic annulus (31). Since the magnetic annulus (31) of the resultant magnetic field (3) is generated by the like poles of the two magnets (5), a head portion of the magnetic member (12), when entering the magnetic annulus (31), is repelled due to repulsion between the two like poles and in turn drives the multi joint section (11) to bend in the direction of the bending degree of freedom, making the free end of the multi joint section (11) advance in the desired direction (D).

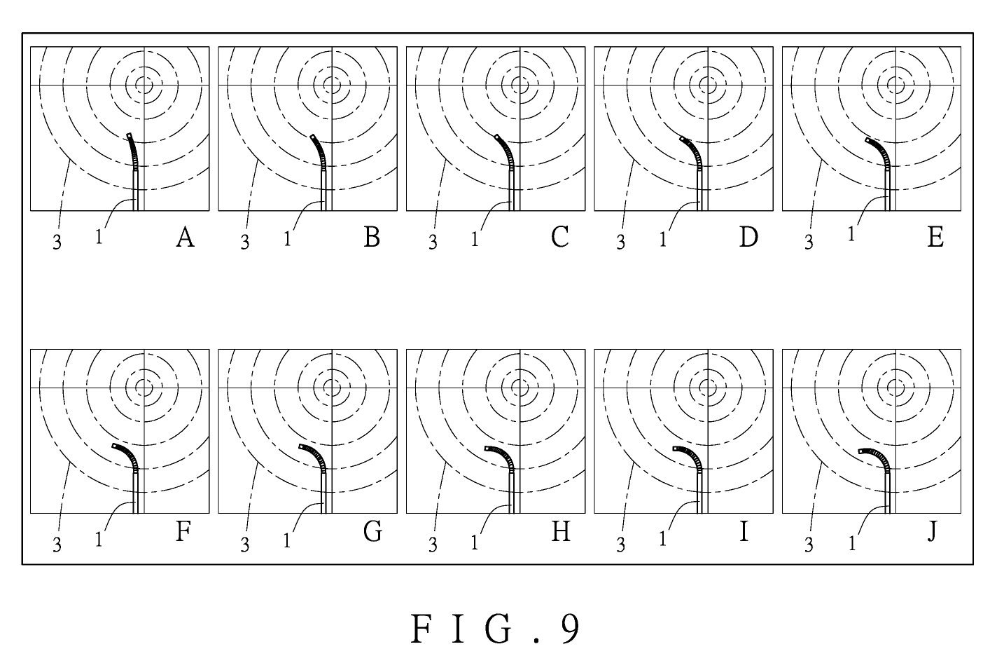

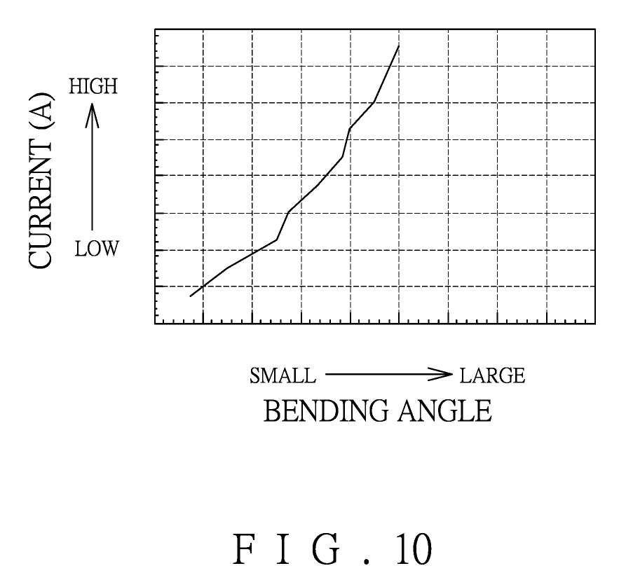

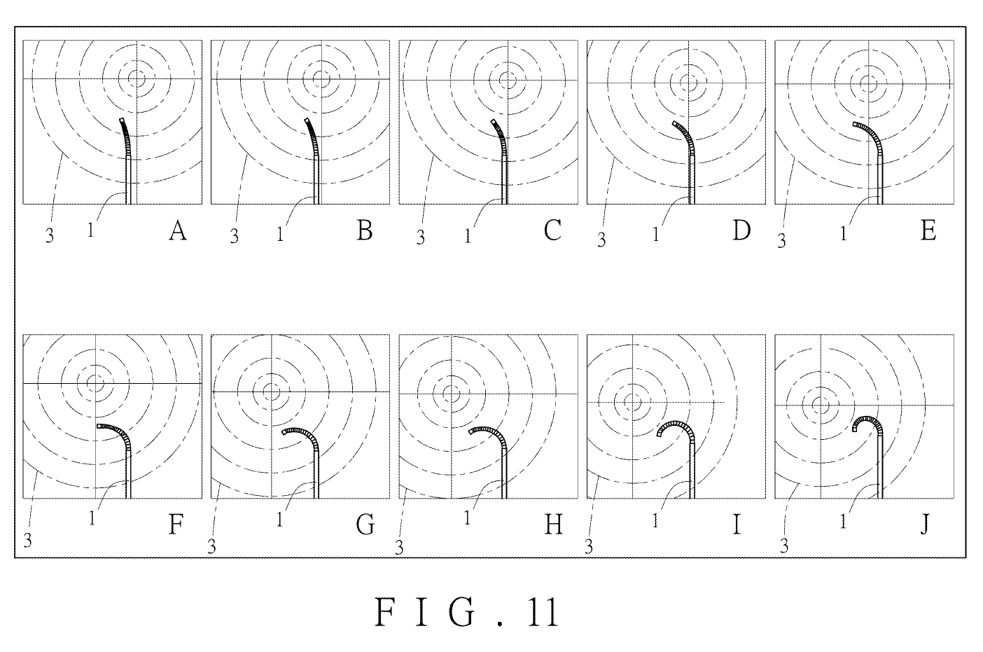

Referring to FIG. 9 and FIG. 10, thanks to its structure or/and material, the multi joint section (11) is resilient and, therefore, can generate a resilient returning force against the magnetic force. Therefore, when the multi joint section (11) performs the bending motion, the currents for the electromagnets (5) have to be gradually increased, so as to make the strength of the resultant magnetic field (3) gradually increase, thereby increasing the angle on which the multi joint section (11) bends. Further, in the case that the two opposite electromagnets (5) are not moved synchronously when the multi joint section (11) performs the bending motion, higher currents for the electromagnets (5) might be required in order to enhance the magnetic force because the magnetic member (12) of the multi joint section (11) could enter a region having lower magnetic flux density than the magnetic annulus (31).

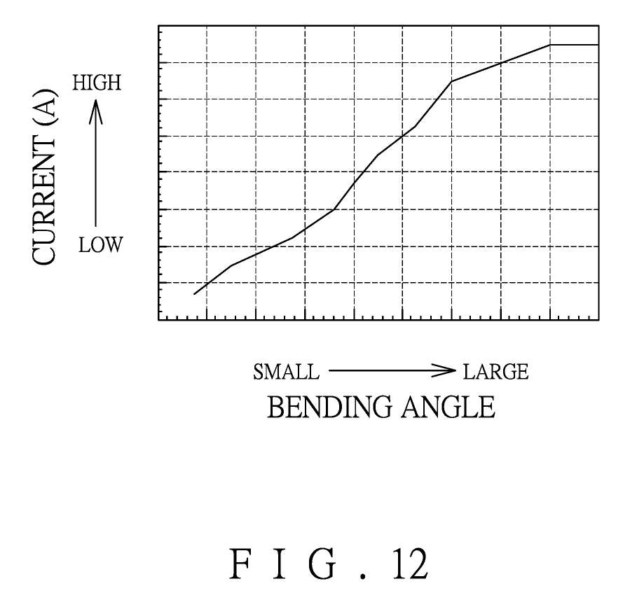

Referring to FIG. 11 and FIG. 12, in the event that the magnetic member (12) of the multi joint section (11) is retained in the magnetic annulus by moving the two electromagnets (5) while the multi joint section (11) performs the bending motion, the multi joint section (11) can perform the bending motion easier and even bend by a larger angle, thanks to the repulsion between the like poles of the two electromagnets (5). Moreover, the multi joint section (11) is bendable in the direction perpendicular to an extending direction of the two electromagnets (5) and is allowed to bend in multiple directions in the case that each joint thereof has a respective bending degree of freedom which is different from another joint thereof.

Referring to FIG. 10 and FIG. 12, when the currents for the two electromagnets (5) are fixed, a wider bending angle can be achieved in the case that the two electromagnets (5) are moved synchronously with the multi joint section (11), in comparison to the case that the two electromagnets (5) are not moved synchronously.

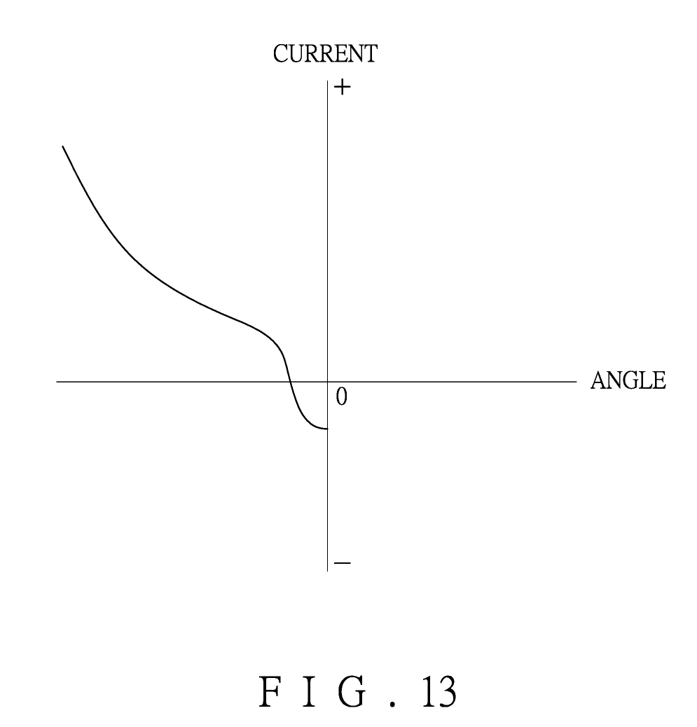

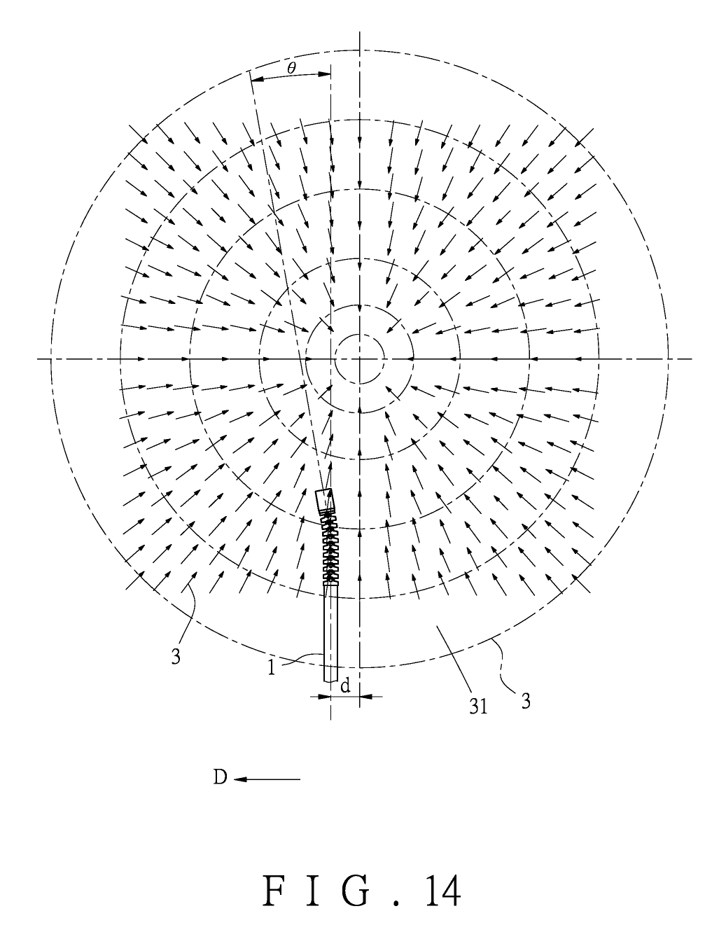

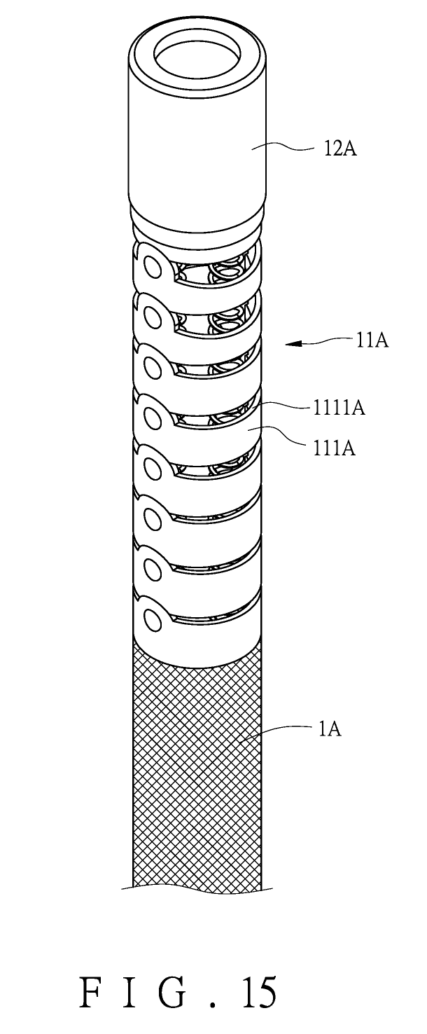

Referring to FIG. 13 and FIG. 14, for making the multi joint section (11) return to its initial state, the currents for the electromagnets (5) are gradually reduced, so as to make the strength of the resultant magnetic field (3) gradually decrease, thereby lowering the thrust acting on the multi joint section (11) and allowing the multi joint section (11) to gradually return due to its own resilient returning force. As a result of a natural physical phenomenon, the multi joint section (11) is not directly returned to its initial extending direction. Thus, after the multi joint section (11) has returned to a preset angle (0), such as an angle between 10 and 30 degrees, the resultant magnetic field (3) is reversed so as to change the direction of the magnetic force and generate a pull force applied on the multi joint section (11), thereby making the multi joint section (11) return to its initial state exactly.

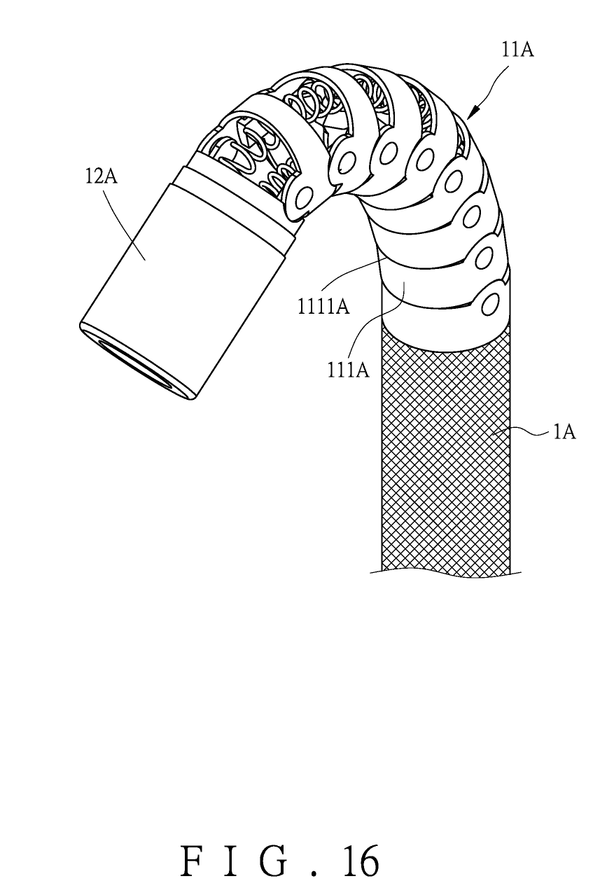

Referring to FIG. 15 and FIG. 16, for making the bending motion of the disclosed magnetic catheter meets the requirement of clinical use, an exemplificative structure of an endoscopic catheter (1A) is proposed.

The magnetic catheter (1A) has a front end provided with a multi joint section (11A) with each joint (111A) thereof having a single bending degree of freedom. At a free end of the multi joint section (11A), there is a magnetic member (12A). The joints (111A) of the multi joint section (11A) are pivotally connected one by one. Each of two adjacent said joints (111A) has an inclined abutting surface (1111A) that faces the inclined abutting surface (1111A) of the other, so that when the multi joint section (11A) performs the bending motion in the direction of the bending degree of freedom, the abutting surfaces (1111A) of each two adjacent said joints (111A) abut on each other. Preferably, the joint (111A) closer to the free end has its abutting surface (1111A) inclined more. In addition, among the joints (111A) of the multi joint section (11A), the one closer to the free end is shorter.

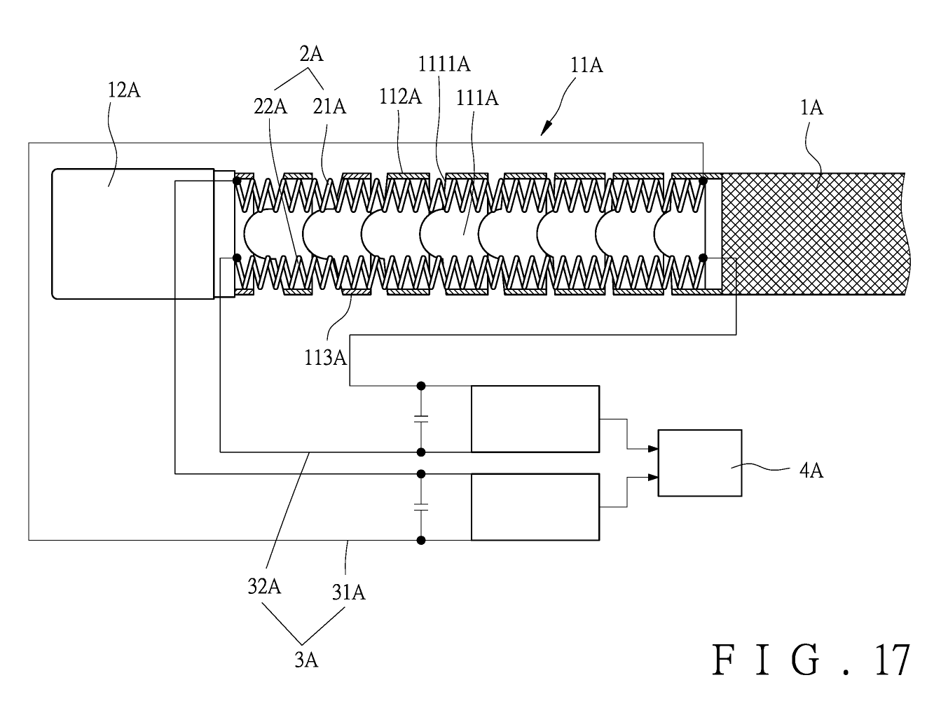

Referring to FIG. 17, furthermore, an elastic unit (2A) is provided between the two ends of the multi joint section (11A). When the multi joint section (11A) performs the bending motion, the elastic unit (2A) performs elastic deformation accordingly.

Referring to FIG. 17, the multi joint section (11A) has a first side (112A) and a second side (113A) opposite to the first side (112A). The bending degree of freedom of the multi joint section (11A) allows the multi joint section (11A) to bend toward either the first side (112A) or the second side (113A). In the present embodiment, the elastic unit (2A) comprises a first elastic member (21A) combined with the first side (112A) and a second elastic member (22A) combined with the second side (113A). There are also a sensing circuit (3A) and a processing unit (4A) that is electrically connected to the sensing circuit (3A). The sensing circuit (3A) comprises a first sensing circuit (31A) connected to the first elastic member (21A), and a second sensing circuit (32A) connected to the second elastic member (22A).

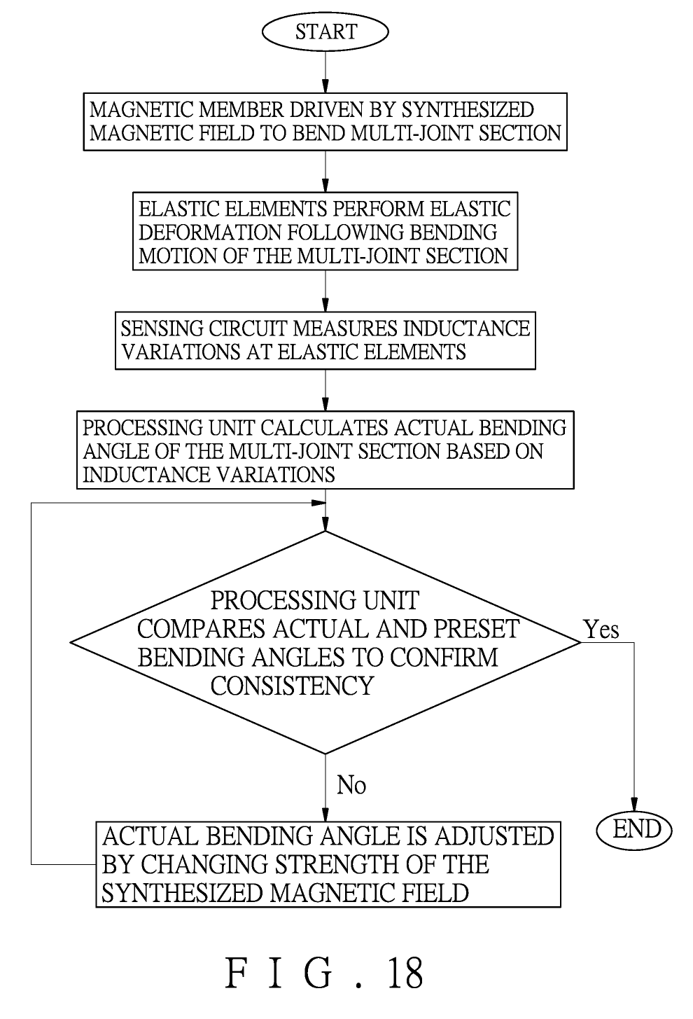

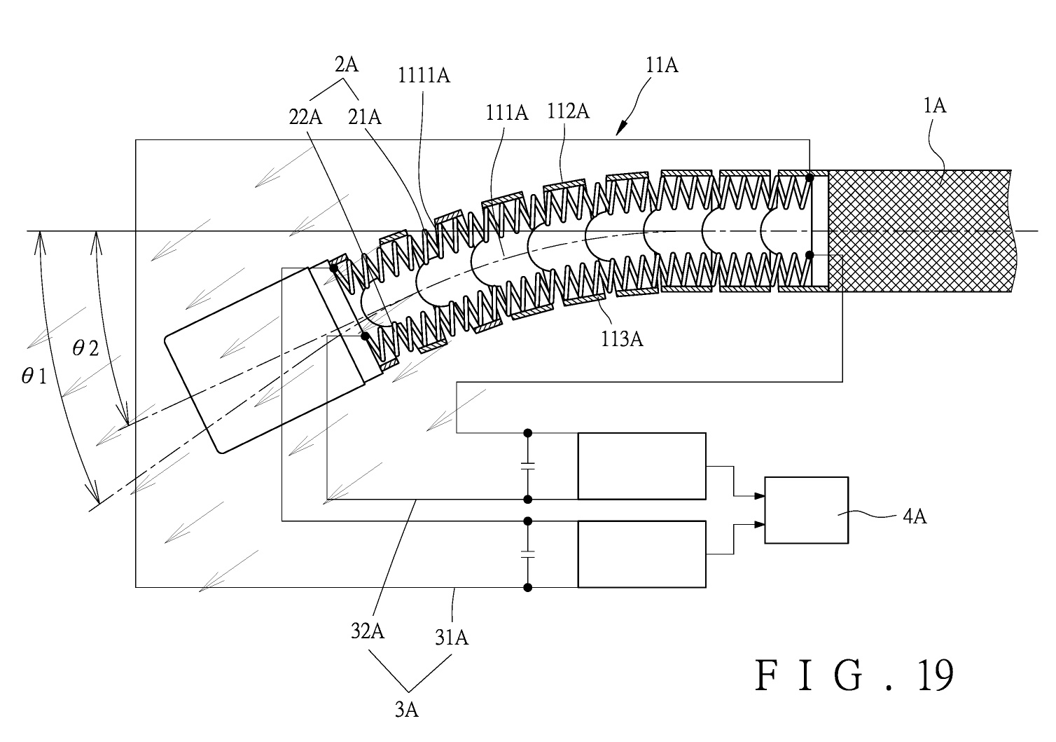

Now the reference is made to FIG. 18 and FIG. 19.

In Step A, the strength of the resultant magnetic field (3) is set according to a preset bending angle (θ1), and the magnetic member (12A) is thus driven by the resultant magnetic field (3) to make the multi joint section (11A) perform the bending motion. As used herein, the preset bending angle (θ1) refers to an angle on which the multi joint section (11A) bends in a patient's body as expected by doctors, i.e., in a direction aligning with nidi. By reaching the preset bending angle (θ1), the multi joint section (11A) can make a certain target site visible and accessible to the doctors. When the resultant magnetic field (3) makes the multi joint section (11A) bend from the first side (112A) toward the second side (113A), the first elastic member (21A) performs elastic deformation and elongates, while the second elastic member (22A) performs elastic deformation and contracts. The first sensing circuit (31A) measures variation of the inductance value caused by the elongation of the first elastic member (21A) at the first side (112A), and the second sensing circuit (32A) measures variation of the inductance value caused by the contraction of the second elastic member (22A) at the second side (113A).

In Step B, the variation of the inductance value are input to the processing unit (4A), and the processing unit (4A) uses this information to calculate an actual bending angle (θ2) of the multi joint section (11A). Since the variations of the inductance values include the variation of the inductance value caused by the elongation of the first elastic member (21A), and the variation of the inductance value caused by the contraction of the second elastic member (22A), the processing unit (4A) can also use this information to determine whether the multi joint section (11A) correctly bends from the first side (112A) toward the second side (113A).

In Step C, the actual bending angle (θ2) and the preset bending angle (θ1) are compared so the doctors can determine whether the actual bending angle (θ2) coincides with the preset bending angle (θ1). If there is any inconsistency therebetween, this informational also enables the doctors to adjust the actual bending angle (θ2) of the multi joint section (11A) until the actual bending angle (θ2) becomes equal to the preset bending angle (θ1) which means the multi joint section (11A) advances toward the direction aligning with nidi.

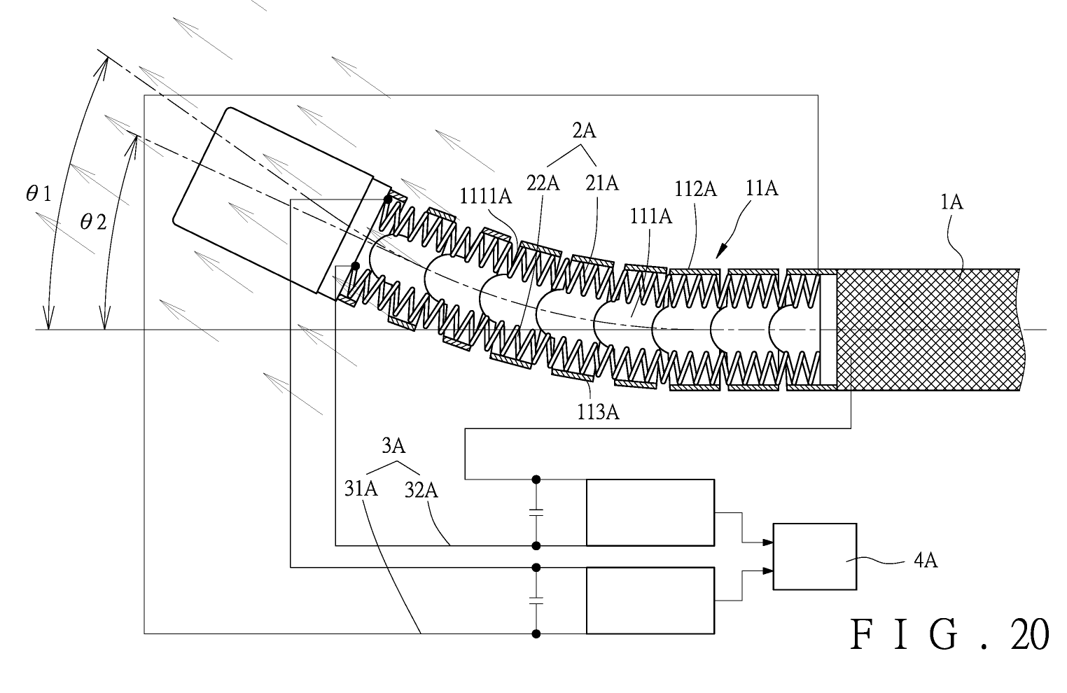

As shown in FIG. 20, when the multi joint section (11A) bends from the second side (113A) toward the first side (112A), the first elastic member (21A) is contracted due to elastic deformation while the second elastic member (22A) is elongated due to elastic deformation. The first sensing circuit (31A) measures variation of the inductance value caused by the contraction of the first elastic member (21A) at the first side (112A), and the second sensing circuit (32A) measures variation of the inductance value caused by the elongation of the second elastic member (22A) at the second side (113A). The subsequent comparison, determination and adjustment are similar to the previous embodiments and are not discussed in any length herein.

The present invention has been described with reference to the preferred embodiments and it is understood that the embodiments are not intended to limit the scope of the present invention. Moreover, as the contents disclosed herein should be readily understood and can be implemented by a person skilled in the art, all equivalent changes or modifications which do not depart from the concept of the present invention should be encompassed by the appended claims.

What is claimed is:

1. A method for controlling a magnetic catheter by using a magnetic-field-generated magnetic annulus, the magnetic catheter being provided with a magnetic member, and the method comprising the following steps:
generating a resultant magnetic field between at least two magnets, wherein an annular region that has a relative high magnetic flux density in the resultant magnetic field is defined as the magnetic annulus;
entering the magnetic catheter into the magnetic annulus so that a magnetic force is produced by the interaction between the magnetic member and the magnetic annulus; and
driving the magnetic member by the magnetic force in order to lead the magnetic catheter to perform a preset motion.

2. The method of claim 1, wherein the magnetic annulus of the resultant magnetic field is generated by arranging the at least two magnets with their like poles facing each other.

3. The method of claim 2, wherein the magnetic catheter has a front section that is a flexible section, in which the flexible section and a rear section of the magnetic catheter have different rigidities due to the fact that they are made of different materials; the flexible section has a free end provided with the magnetic member; the preset motion is to make the flexible section perform a bending motion.

4. The method of claim 3, wherein the flexible section is resilient and therefore able to generate a resilient returning force, and the method further comprises when the flexible section performs the bending motion, gradually increasing or decreasing a strength of the resultant magnetic field in a certain ratio according to the resilient returning force generated, so as to adjust the magnetic force of the magnetic annulus and control a bending angle of the flexible section.

5. The method of claim 4, further comprising when the flexible section has performed the bending motion, gradually decreasing the strength of the resultant magnetic field, thereby reducing the magnetic force, so that the resilient returning force gradually returns the flexible section, and comprises when the flexible section returns to a set angle, reversing the resultant magnetic field, thereby making the flexible section return to an initial state thereof under a magnetic force.

6. The method of claim 3, further comprising varying, when the flexible section performs the bending motion, the resultant magnetic field according to a certain target site and retaining the magnetic member within the magnetic annulus, wherein said varying the resultant magnetic field includes changing position or/and direction or/and strength of the resultant magnetic field,.

7. The method of claim 6, wherein changing position or/and direction or/and strength of the resultant magnetic field is achieved by moving the at least two magnets.

8. The method of claim 6, wherein the at least two magnets are electromagnets, and wherein changing position or/and direction or/and strength of the resultant magnetic field is achieved by changing current intensities or/and current directions of the electromagnets.

9. The method of claim 6, further comprising controlling the magnetic catheter to perform feeding motion or/and rotating motion while the flexible section performs the bending motion.

10. The method of claim 2, wherein the magnetic catheter has a front section that is a flexible section, in which the flexible section is a multi joint section; the flexible section has a free end provided with the magnetic member; the preset motion is to make the flexible section perform a bending motion.

11. The method of claim 10, wherein each joint of the multi joint section has a single bending degree of freedom, and the preset motion is to make the multi joint section perform the bending motion in a direction of the bending degree of freedom.

12. The method of claim 10, wherein the flexible section is resilient and therefore able to generate a resilient returning force, and the method further comprises when the flexible section performs the bending motion, gradually increasing or decreasing a strength of the resultant magnetic field in a certain ratio according to the resilient returning force generated, so as to adjust the magnetic force of the magnetic annulus and control a bending angle of the flexible section.

13. The method of claim 12, further comprising gradually decreasing, when the flexible section has performed the bending motion, the strength of the resultant magnetic field, thereby reducing the magnetic force, so that the resilient returning force gradually returns the flexible section, and further comprising reversing, when the flexible section returns to a set angle, the resultant magnetic field, thereby making the flexible section return to an initial state thereof under a magnetic force.

14. The method of claim 10, further comprising varying, when the flexible section performs the bending motion, the resultant magnetic field according to a certain target site and retaining the magnetic member within the magnetic annulus, wherein said varying the resultant magnetic field includes changing position or/and direction or/and strength of the resultant magnetic field.

15. The method of claim 14, wherein changing position or/and direction, or/and strength of the resultant magnetic field is achieved by moving the at least two magnets.

16. The method of claim 14, wherein the at least two magnets are electromagnets, and wherein changing position or/and direction or/and strength of the resultant magnetic field is achieved by changing current intensities or/and current directions of the electromagnets.

17. The method of claim 14, further comprising feeding or/and rotating the magnetic catheter while the flexible section performs the bending motion.

18. The method of claim 1, wherein the magnetic member is an axial magnet.

19. The method of claim 1, wherein the at least two magnets are permanent magnets or electromagnets.

20. The method of claim 1, wherein the magnetic catheter is a flexible endoscope.
